(19) United States
(12) Patent Application Publication
(10) Pub. No.: US 2011/0029041 A1
(43) Pub. Date: Feb. 3, 2011

(54) HEARING PROSTHESIS WITH AN IMPLANTABLE MICROPHONE SYSTEM

(76) Inventor: Pieter Wiskerke, Antwerpen (BE)

Correspondence Address: JOHN S. PRATT, ESQ. (Cochlear);KILPATRICK STOCKTON LLP, 1100 PEACHTREE STREET, SUITE 2800, ATLANTA, GA, 30309, US

(21) Appl. No.: 12/847,871

(22) Filed: Jul. 30, 2010

(30) Foreign Application Priority Data
Jul. 30, 2009 (DE) 102009035386.0

(51) Int. Cl. A61F 11/04; A61N 1/36

(52) U.S. Cl. 607/57

(57) ABSTRACT

A hearing prosthesis including an implantable housing containing a detector, the hearing prosthesis further including a light source, a fiber optical waveguide extending from the implantable housing in light communication with the light source and the detector, and an interferometer connected to the fiber optical waveguide and located outside of the implantable housing, the interferometer being in light communication with the fiber optical waveguide, the light source and the detector. The detector is configured to convert a light signal indicative of acoustic energy impinging upon the interferometer into an electrical signal indicative of the acoustic energy. In an exemplary embodiment, the electrical signal is used by a sound processor of the hearing prosthesis to enhance hearing.

### CROSS-REFERENCE TO RELATED APPLICATIONS

This Patent Application claims priority from German Patent Application No. DE 10 2009 035 386.0, entitled “Hearing Aid Implant,” filed on 30 Jul., 2009, which is hereby incorporated by reference herein.

### BACKGROUND

1. Field of the Invention

The present invention relates generally to hearing prostheses and, more particularly, to an implantable microphone system.

2. Related Art

Medical devices having one or more implantable components, generally referred to as implantable medical devices, have provided a wide range of therapeutic benefits to patients over recent decades. In particular, implanted devices such as hearing aids, pacemakers, defibrillators, functional electrical stimulation devices, cochlear prostheses (also referred to herein as cochlear implants), organ assist or replacement devices, and other partially or completely-implanted medical devices, have been successful in performing life saving and/or lifestyle enhancement functions for a number of years.

Many implantable components receive power and/or data from external components that are part of, or operate in conjunction with, the implantable component. For example, some implantable medical devices include a power source integrated into the implantable component.

A cochlear prosthesis is a specific type of hearing prostheses that delivers electrical stimulation to the recipient's cochlea. As used herein, cochlear implants also include hearing prostheses that deliver electrical stimulation in combination with other types of stimulation, such as acoustic or mechanical stimulation.

Hearing prostheses often utilize microphones to sense or otherwise detect sound waves and convert the detected sound waves into an electrical signal indicative of the sound waves for use by the hearing prostheses. These microphones may be located at various locations on the recipient.

### SUMMARY

According to a first aspect of the present invention, there is a hearing prosthesis including an implantable housing containing a detector, the hearing prosthesis further including a light source, a fiber optical waveguide extending from the implantable housing in light communication with the light source and the detector, and an interferometer connected to the fiber optical waveguide and located outside of the implantable housing, the interferometer being in light communication with the fiber optical waveguide, the light source and the detector. The detector is configured to convert a light signal indicative of acoustic energy impinging upon the interferometer into an electrical signal indicative of the acoustic energy.

According to another aspect of the present invention, there is an implantable Fabry-Perot interferometer configured for use in at least one of a microphone and a vibration sensor, the Fabry-Perot interferometer comprises a base with a cavity, a semi-reflective surface that is located in the cavity and a reflective surface that is located in the cavity spaced apart from the semi-reflective surface, wherein at least one of the semi-reflective surface and the reflective surface is a flexible diaphragm.

According to yet another aspect of the present invention, there is an implantable fixation device adapted for mounting an implantable microphone in a skull bone, wherein the fixation device comprises a first body with an external thread adapted to be screwable into a skull bone of a human and having a conically shaped through hole and a second body that can be secured to the first body, which has a conically shaped end portion, so that the second body fits into the conically shaped through hole of the first body, wherein the implantable microphone is adapted to be embodied into the second body.

### BRIEF DESCRIPTION OF THE DRAWINGS

Embodiments of the present invention are described in the following detailed description when taken with reference to the accompanying drawings in which:

FIG. 1 is a perspective view of an exemplary totally implantable cochlear implant, in which embodiments of the present invention may be implemented;

FIG. 2 depicts a principle of an optical microphone according to an exemplary embodiment of the present invention;

FIG. 3 depicts the implantable main housing containing a driving and sensing device, the signal processor and antennae according to an exemplary embodiment of the present invention;

FIG. 4 depicts a Fabry-Perot microphone according to an exemplary embodiment of the present invention;

FIG. 5 depicts an implantation configuration according to an exemplary embodiment of the present invention;

FIG. 6 depicts a bone fixation of the implantable optical microphone according to an exemplary embodiment of the present invention;

FIGS. 7 A and 7 B depict a modification of the Fabry-Perot microphone of FIG. 4 and its working principle during operation according to an exemplary embodiment of the present invention;

FIGS. 8A and 8B depicts two alternative examples of implanted configurations with two sensors instead of one sensor, as is shown in FIG. 5;

FIGS. 9A and 9B depict further implantation configurations of the hearing prosthesis according to an exemplary embodiment of the present invention, with the sensor implanted at different locations;

FIGS. 10A and 10B depict modifications of the Fabry-Perot device illustrated in FIGS. 7A and 7B;

FIG. 11 depicts an example for implanting optical sensors usable in a configuration according to FIG. 8A;

FIG. 12 depicts an example for implanting optical vibration sensors according to FIGS. 10 A and 10 B usable in a configuration according to FIG. 8B;

FIG. 13 depicts an implanted optical microphone according to the state of the art usable in combination with a configuration according to FIG. 8B;

FIG. 14 depicts an alternative implantation configuration with a percutaneous optical microphone according to the present disclosure.

FIG. 15 is a functional block diagram of a totally implant cochlear implant in accordance with embodiments of the present invention shown with an external device.

### DETAILED DESCRIPTION

An embodiment of the present invention includes a hearing prosthesis including an implantable housing containing a detector in light communication with an interferometer exterior to the implantable housing. Embodiments further include an interferometer including flexible components, including flexible diaphragms that flex in response to the impingement of sound waves thereon. Embodiments also include a mounting fixture to subcutaneously mount the interferometer in bone of a recipient.

An exemplary embodiment of the present invention relating to the hearing prosthesis including an implantable housing includes the feature that all of the electronic components of an implantable microphone system are hermetically sealed within the implantable housing.

An embodiment of the present invention includes a hearing prosthesis including an implantable housing containing a detector, the hearing prosthesis further including a light source, a fiber optical waveguide extending from the implantable housing in light communication with the light source and the detector, and an interferometer connected to the fiber optical waveguide and located outside of the implantable housing, the interferometer being in light communication with the fiber optical waveguide, the light source and the detector. The detector is configured to convert a light signal indicative of acoustic energy impinging upon the interferometer into an electrical signal indicative of the acoustic energy. In an exemplary embodiment, the electrical signal is used by a sound processor of the hearing prosthesis to enhance hearing. By way of example, a sound processor of a cochlear implant may base cochlear electrode array stimulation signals on the electrical signal.

Embodiments of the present invention include a hearing prosthesis including an implantable interferometer and a fixation device for fixing the implantable interferometer in a recipient. The implantable interferometer may be configured so that it can be used as an optical microphone or an optical vibration sensor or a combination thereof. The implantable interferometer may be of the type wherein two parallel reflecting mirrors are used, the distance of which is influenced by impinged sound or vibrations so that the transmission properties of the two parallel mirrors vary dependent on that distance. This type of interferometer is known as a Fabry-Perot type interferometer.

Embodiments of the present invention are described herein primarily in connection with one type of implantable medical device, a hearing prosthesis, namely a cochlear prosthesis (commonly referred to as cochlear prosthetic devices, cochlear implants, cochlear devices, and the like; simply “cochlear implants” herein.) Cochlear implants deliver electrical stimulation to the cochlea of a recipient. It should, however, be understood that the current techniques described herein are also applicable to other types of active implantable medical devices (AIMDs), such as, auditory brain stimulators, also sometimes referred to as an auditory brainstem implant (ABI), other implanted hearing aids or hearing prostheses, neural stimulators, retinal prostheses, cardiac related devices such as pacers (also referred to as pacemakers) or defibrillators, implanted drug pumps, electro-mechanical stimulation devices (e.g., direct acoustic cochlear stimulators (DACS)) or other implanted electrical devices.

As used herein, cochlear implants also include hearing prostheses that deliver electrical stimulation in combination with other types of stimulation, such as acoustic or mechanical stimulation (sometimes referred to as mixed-mode devices). It would be appreciated that embodiments of the present invention may be implemented in any cochlear implant or other hearing prosthesis now known or later developed, including auditory brain stimulators, or implantable hearing prostheses that mechanically stimulate components of the recipient's middle or inner ear. For example, embodiments of the present invention may be implemented, for example, in a hearing prosthesis that provides mechanical stimulation to the middle ear and/or inner ear of a recipient.

Embodiments of the present invention are further described herein primarily in connection with one type of cochlear prosthesis, namely a totally or fully implantable cochlear prosthesis. As used herein, a totally implantable cochlear implant refers to an implant that is capable of operating, at least for a period of time, without the need for any external device. It would be appreciated that embodiments of the present invention may also be implemented in a cochlear implant that includes one or more external components. It would be further appreciated that embodiments of the present invention may be implemented in any partially or fully implantable hearing prosthesis now known or later developed, including, but not limited to, acoustic hearing aids, auditory brain stimulators, middle ear mechanical stimulators, hybrid electro-acoustic prosthesis or other prosthesis that electrically, acoustically and/or mechanically stimulate components of the recipient's outer, middle or inner ear or in which it may be useful to align an external device with an implanted component.

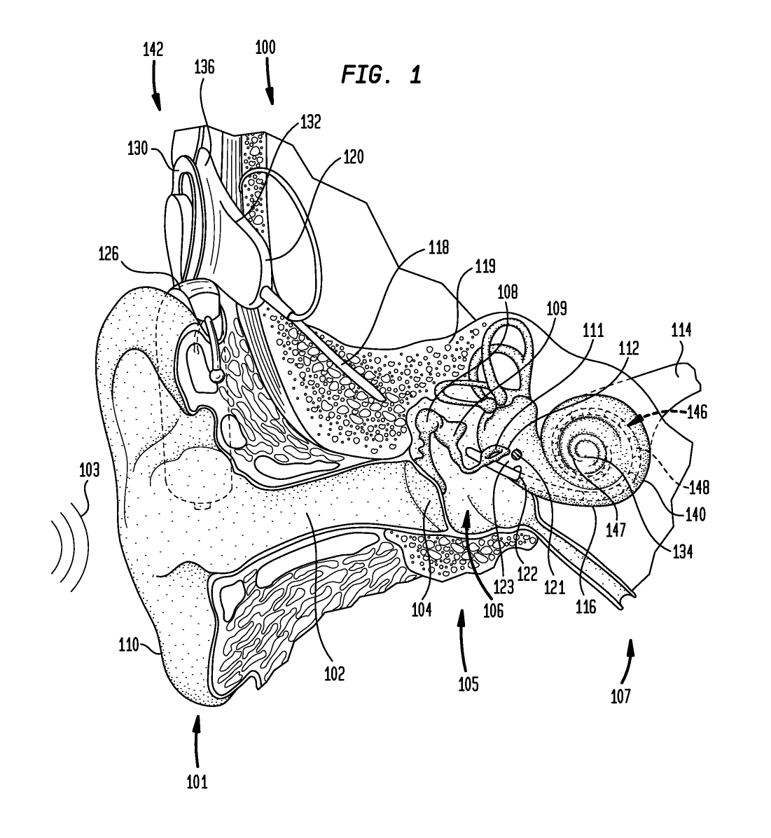

FIG. 1 is perspective view of a totally implantable cochlear implant, referred to as cochlear implant 100, implanted in a recipient. The recipient has an outer ear 101, a middle ear 105 and an inner ear 107. Components of outer ear 101, middle ear 105 and inner ear 107 are described below, followed by a description of cochlear implant 100.

In a fully functional ear, outer ear 101 comprises an auricle 110 and an ear canal 102. An acoustic pressure or sound wave 103 is collected by auricle 110 and channeled into and through ear canal 102. Disposed across the distal end of ear cannel 102 is a tympanic membrane 104 which vibrates in response to sound wave 103. This vibration is coupled to oval window or fenestra ovalis 112 through three bones of middle ear 105, collectively referred to as the ossicles 106 and comprising the malleus 108, the incus 109 and the stapes 111. Bones 108, 109 and 111 of middle ear 105 serve to filter and amplify sound wave 103, causing oval window 112 to articulate, or vibrate in response to vibration of tympanic membrane 104. This vibration sets up waves of fluid motion of the perilymph within cochlea 140. Such fluid motion, in turn, activates tiny hair cells (not shown) inside of cochlea 140. Activation of the hair cells causes appropriate nerve impulses to be generated and transferred through the spiral ganglion cells (not shown) and auditory nerve 114 to the brain (also not shown) where they are perceived as sound.

As shown, cochlear implant 100 comprises one or more components which are temporarily or permanently implanted in the recipient. Cochlear implant 100 is shown in FIG. 1 with an external device 142 which, as described below, is configured to provide power to the cochlear implant.

In the illustrative arrangement of FIG. 1, external device 142 may comprise a power source (not shown) disposed in a Behind-The-Ear (BTE) unit 126. External device 142 also includes components of a transcutaneous energy transfer link, referred to as an external energy transfer assembly. The transcutaneous energy transfer link is used to transfer power and/or data to cochlear implant 100. As would be appreciated, various types of energy transfer, such as infrared (IR), electromagnetic, capacitive and inductive transfer, may be used to transfer the power and/or data from external device 142 to cochlear implant 100. In the illustrative embodiments of FIG. 1, the external energy transfer assembly comprises an external coil 130 that forms part of an inductive radio frequency (RF) communication link. External coil 130 is typically a wire antenna coil comprised of multiple turns of electrically insulated single-strand or multi-strand platinum or gold wire. External device 142 also includes a magnet (not shown) positioned within the turns of wire of external coil 130. It should be appreciated that the external device shown in FIG. 1 is merely illustrative, and other external devices may be used with embodiments of the present invention.

Cochlear implant 100 comprises an internal energy transfer assembly 132 which may be positioned in a recess of the temporal bone adjacent auricle 110 of the recipient. As detailed below, internal energy transfer assembly 132 is a component of the transcutaneous energy transfer link and receives power and/or data from external device 142. In the illustrative embodiment, the energy transfer link comprises an inductive RF link, and internal energy transfer assembly 132 comprises a primary internal coil 136. Internal coil 136 is typically a wire antenna coil comprised of multiple turns of electrically insulated single-strand or multi-strand platinum or gold wire. Positioned substantially within the wire coils is an implantable microphone system (not shown). As described in detail below, the implantable microphone assembly includes a microphone (not shown), and a magnet (also not shown) fixed relative to the internal coil.

Cochlear implant 100 further comprises a main implantable component 120 and an elongate electrode assembly 118. In embodiments of the present invention, internal energy transfer assembly 132 and main implantable component 120 are hermetically sealed within a biocompatible housing. In embodiments of the present invention, main implantable component 120 includes a sound processing unit (not shown) to convert the sound signals received by the implantable microphone in internal energy transfer assembly 132 to data signals. Main implantable component 120 further includes a stimulator unit (also not shown) which generates electrical stimulation signals based on the data signals. The electrical stimulation signals are delivered to the recipient via elongate electrode assembly 118.

Elongate electrode assembly 118 has a proximal end connected to main implantable component 120, and a distal end implanted in cochlea 140. Electrode assembly 118 extends from main implantable component 120 to cochlea 140 through mastoid bone 119. In some embodiments electrode assembly 118 may be implanted at least in basal region 116, and sometimes further. For example, electrode assembly 118 may extend towards apical end of cochlea 140, referred to as cochlea apex 134. In certain circumstances, electrode assembly 118 may be inserted into cochlea 140 via a cochleostomy 122. In other circumstances, a cochleostomy may be formed through round window 121, oval window 112, the promontory 123 or through an apical turn 147 of cochlea 140.

Electrode assembly 118 comprises a longitudinally aligned and distally extending array 146 of electrodes 148, sometimes referred to as electrode array 146 herein, disposed along a length thereof Although electrode array 146 may be disposed on electrode assembly 118, in most practical applications, electrode array 146 is integrated into electrode assembly 118. As such, electrode array 146 is referred to herein as being disposed in electrode assembly 118. As noted, a stimulator unit generates stimulation signals which are applied by electrodes 148 to cochlea 140, thereby stimulating auditory nerve 114.

As noted, cochlear implant 100 comprises a totally implantable prosthesis that is capable of operating, at least for a period of time, without the need for external device 142. Therefore, cochlear implant 100 further comprises a rechargeable power source (not shown) that stores power received from external device 142. The power source may comprise, for example, a rechargeable battery. During operation of cochlear implant 100, the power stored by the power source is distributed to the various other implanted components as needed. The power source may be located in main implantable component 120, or disposed in a separate implanted location.

Additional features of the cochlear implant 100 are described below with reference to functional diagrams of the cochlear implant 100 according to an embodiment of the present invention. Before this, however, exemplary embodiments of the implantable microphone that may be utilized with cochlear implant 100 will now be described. It is noted that while cochlear implant 100 has been disclosed as a totally implantable cochlear prosthesis, embodiments of the implantable microphone may be practiced with a cochlear implant that is not totally implantable.

It is noted that embodiments of the present invention may be practiced with implants other than cochlear implants, such as, for example, implanted heart monitors, implanted muscle stimulation devices, etc. Accordingly, an embodiment of the present invention will first be described in the context of a non-descript implant, and later, embodiments of the present invention will be described in the context of a cochlear implant.

In an exemplary embodiment of the present invention that utilizes an implantable microphone, the implantable microphone is biocompatible and hermetically sealed. As will be described in greater detail below, an embodiment of the present invention includes implanting the implantable microphone under a layer of skin or soft tissue such that the overlying skin or soft tissue acts to attenuate air carried sound signals, through reflection, scattering and/or absorption.

An embodiment of the present invention alleviates some or all of the loss of signal that occurs through impedance matching effects associated with the sound signal passing from air into the body. An embodiment of the present invention also reduces and/or eliminates the internal body noise that may be detected by the microphone and/or conveyed by the microphone to a processor that processes the outputted signal from the microphone. An implantable microphone can, for example, be constructed by coupling a transducer (electric, piezo or other pressure sensitive transducer) to the solid floor of the cavity covered by a titanium diaphragm.

An embodiment of the present invention includes implanting an implantable microphone such that bone conducted body noise detected by the microphone and/or conveyed by the microphone to a processor that processes the outputted signal from the microphone is reduced and/or eliminated. In an embodiment, the implanted microphone reduces and/or eliminates the effects of acceleration of the implant package (e.g., the implantable microphone) against the skin. In an embodiment, the effects of mass loading on the implantable microphone vis-à-vis sensitivity of the microphone to vibration on the microphone are reduced and or eliminated. In an embodiment of the present invention, the implantable microphone is implanted to harness, at least in part, natural amplification of noise or sound that occurs in the human ear. In an embodiment, there is a subcutaneous microphone that is used in a system such that directionality cues are given to the recipient. In yet another embodiment of the present invention, the implantable microphone is relatively resistant to impact.

An embodiment of the present invention includes a subcutaneous implantable microphone that is placed in the outer part of the ear canal of a human ear (the outer ear). Such a system utilizes natural amplification (5-20 dB dependent on frequency and direction) of noise/sound waves received by the human ear, and permits directionality of the noise/sound waves to be perceived. In another embodiment of the present invention, there is an implantable microphone system that reduces and/or eliminates the likelihood of skin necrosis and cholesteatoma resulting from the implantation of the implantable microphone.

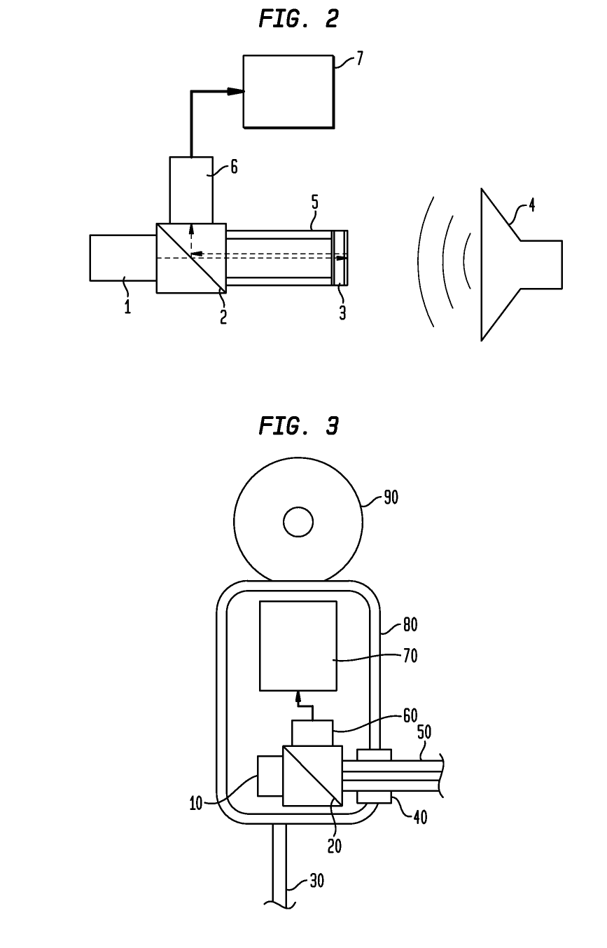

Referring now to FIG. 2, there is an implantable microphone system including an optical microphone according to an embodiment of the present invention. It is noted at this time that the term “microphone” as used herein includes devices configured to sense sound waves and energy waves resulting from sound waves, collectively referred to as acoustic energy waves. FIG. 2 depicts an optical microphone that includes a light source 1 and an optical element 2 that may be an optical directional coupler or a beam splitter. The optical microphone of FIG. 2 includes an interferometer device 3 to which in the following is referred sometimes referred to as a sensor or a sensor part. FIG. 2 depicts a source of sound 4, a fiber optical waveguide 5, a detector 6, and a sound processor 7. It is noted that the sound processor 7 may correspond to the sound processor, etc., of FIG. 1 detailed above. Light emitted from the light source 1 is coupled via the optical element 2 into the fiber optical waveguide 5 and is transmitted to the interferometer device 3 attached at the end of the fiber optical waveguide 5. The emitted light is reflected in/at the interferometer device 3 and is guided through the fiber optical waveguide 5 back to the optical element 2 which directs the reflected light to the detector 6. The detector 6 converts the light signal into an electrical signal which is directed to the sound processor 7.

The sound that is detected/received by the interferometer device 3 initially causes a surface of the interferometer device 3 to vibrate and, in an exemplary embodiment, changes the phase relation between the incident light on the interferometer device 3 and the reflected light from the interferometer device 3. This leads to an intensity modulation of the light reflected in/from the interferometer device 3 which can be detected by detector 6.

The fiber optical waveguide 5 connects light source 1 and detector 6 inside a housing that is configured to be implanted in a recipient (an “implant housing”) with the interferometer device 3 located at the measurement point (also referred to as the sensor point). In an exemplary embodiment, the housing corresponds to the bio-compatible housing described above with respect to FIG. 1.

In an embodiment of the present invention, a laser diode or a vertical-cavity surface emitting laser (VCSEL) may be used as the light source 1. In an embodiment, the light source can be operated in a pulsed operation. Such operation may, in some embodiments, reduce power consumption of the implantable microphone system. In an embodiment, the power consumption may be reduced to approximately 100 μW, and in some embodiments, it may be reduced to even less than that. In some embodiments, the VCSEL and other components, such as a photodetector, etc., may be integrated on a printed circuit board (PCB) and may even be combined with the sound processor on the same PCB, so as to achieve a small size of the implantable housing.

In an embodiment, a very efficient laser diode may be used, such that the diodes provide excellent lasing properties with light having a small bandwidth and high coherence. Moreover, in an embodiment of the present invention utilizing the implantable microphone system as detailed herein, there is a hearing prosthesis that consumes relatively low power amounts. An embodiment includes a VCEL that utilizes vertical emission, thereby permitting mounting of the VCSELs on the integrated printed circuit board.

An embodiment of the present invention includes integrating at least some components of the implantable microphone system into an implantable main housing 80 of a cochlear implant, as is schematically depicted in FIG. 3. FIG. 3 depicts light source 10, optical element 20 (which may be a beam splitter), detector 60, fiber optical waveguide 50, and sound processor 70, which in an embodiment correspond to the respective components presented in FIG. 2, enclosed in an implantable main housing 80. A feedthrough 40 is used to provide a hermetically sealed interface between the fiber optical waveguide 5 and the implantable main housing 80.

In an embodiment, the implantable main housing 80 corresponds to the biocompatible housing, sometimes collectively referred to as a stimulator/receiver unit, detailed above with respect to FIG. 1.

FIG. 3 also depicts an electrical connection 30 corresponding to electrical lead(s) which may connect an electrode array (not shown in FIG. 3) that is implanted in the cochlea of a recipient, or other transducer (e.g., middle ear implant, bone conduction device, conventional hearing aid receiver/speaker)/stimulating device, with the sound processor 70. In an exemplary embodiment, the sound processor 70 processes the electrical sound signals output by the detector 60 and generates signals used to provide stimulation to the cochlear by the electrode array of the cochlear implant/transducer/stimulator.

A high-level example of a hearing prosthesis corresponding to a cochlear implant that utilizes the optical microphone according to an embodiment of the present invention is depicted in FIG. 5. FIG. 5 depicts an implanted main housing 230 with an antenna 240 for transmitting and/or receiving data and energy via a subcutaneous link, and a fiber optical waveguide 220 that connects a driving and detecting unit comprising a light source and a detector within the main housing 230 with the sensor 210, which may correspond to the interferometer device 3 described above with respect to FIG. 2. FIG. 5 further depicts a line 270, which may correspond to electrical leads, that connect the sound processor (shown in FIG. 3) with an electrode array that is inserted in the cochlea.

In an exemplary embodiment, fiber optical waveguide 220 is flexible and has a length of at least about 30, 40, 50, 60, 70, 80, 90 or 100 times the diameter of sensor 210, the diameter of sensor 210 taken on a plane parallel to the diaphragm in contact with the skin/tissue of the recipient.

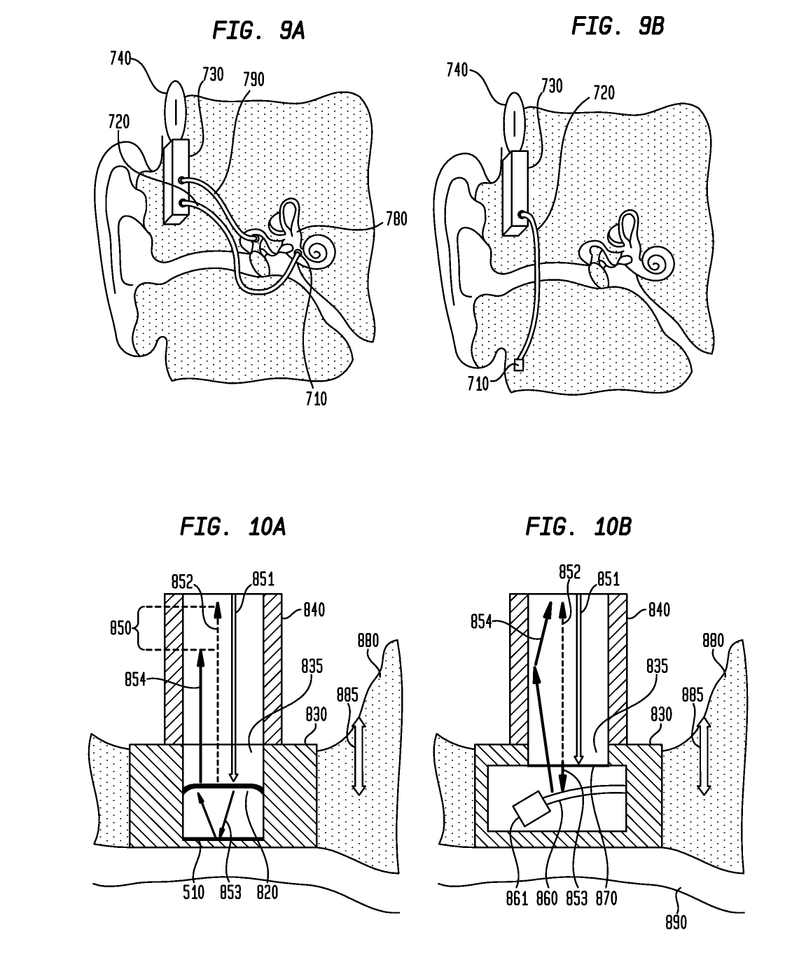

It is noted that the hearing prosthesis of FIG. 5 is an exemplary embodiment of the invention. In another exemplary embodiment, the hearing prosthesis is utilized with devices other than or in addition to cochlear implants. By way of example, the sound processer and corresponding driving electronics stimulation may also be adapted for hybrid hearing devices, MIKI (mostly implanted cochlear implant) and DACS (direct acoustic cochlear stimulation). A DACS configuration is for instance exemplified in FIG. 9A, wherein a line 790 connects driving electronics in the implanted main housing 730 with an actuator that may be attached to one of the ossicles and/or directly to the oval window of the cochlea.

Referring back to FIG. 3, there is shown an antenna 90 which may be used to wirelessly charge an internal battery (not shown) which may be used, at least in part, to provide the electronics of the hearing prosthesis (at least the implantable components) with energy. The antenna 90 may also be used, in an exemplary embodiment, to transmit configuration data and to allow a reconfiguration of the hearing prosthesis and/or for remote control of the implanted components of the hearing prosthesis. Because the implant main housing 80 may be relatively large, and the location where it may be placed in the recipient may be limited, the sensor part (also referred to as the measuring part, which is not show in FIG. 3) of the optical microphone, which may correspond, in some embodiments, to interferometer device 3, may be located outside the main implant housing 80. Thus, an embodiment of the present invention allows for flexibility with respect to the positioning of the sensor (sound receptor), which may correspond to the interferometer device 3, and the sensor may be placed at a location that provides for a relatively good signal to noise ratio and/or provides for other clinical advantages.

Still further, in an exemplary embodiment of the present invention, there is provided a hearing prosthesis that is adapted to separate the bio-toxic materials used on the electronics side of a microphone system from the sensor, which may be biocompatible, because the bio-toxic materials are placed in a hermetically sealed implantable main housing 80.

In an embodiment of the present invention, the sensor may be positioned at a location where it may be vulnerable/more vulnerable to experiencing forces/accelerations resulting from an impact, at least relative to other locations in a recipient. Because an embodiment of the present invention utilizes relatively non or less bio-toxic components, in contrast to the electronic components inside the implantable main housing 80, the likelihood of an unhealthy situation is reduced, relative to a combined microphone system (i.e., where the electronics are co-located with the sensor). That is, in an exemplary embodiment of the present invention, even if the integrity of the bio-compatible sensor according to some embodiments of the present invention is disrupted, there will be little or no harmful effects on the recipient's health. In an exemplary embodiment of the present invention, this is because the fiber optical waveguide 5 itself may be made of a bio-compatible material, such as by way of example, glass fiber or a bio-compatible plastic, etc. The sensor (e.g., the interferometer device 3) may also be made of bio-compatible materials, in an exemplary embodiment, such as, for example, bio-compatible plastic, glass, titanium, tantalum or similar biocompatible materials and compounds.

An exemplary embodiment of the present invention provides for a sensor that is a passive element, and may be adapted so that no electrical leads and converters are needed, this in contrast to conventional microphones. Further, conventional microphones may require analog/digital converters (ADC) to convert direct current into alternating current. Electromagnetic fields due to the alternating current are, however, in some scenarios of use of an implantable microphone, not desirable in/next to living tissues in general, and in particular, close to the brain. Moreover, the analog/digital converters will consume power and may introduce system noise and/or otherwise require additional shielding. These aspects of conventional microphones are at least decreased and/or eliminated in some exemplary embodiments of the present invention, at least when an optical microphone is utilized with a remote sensor part that does not require electrical current.

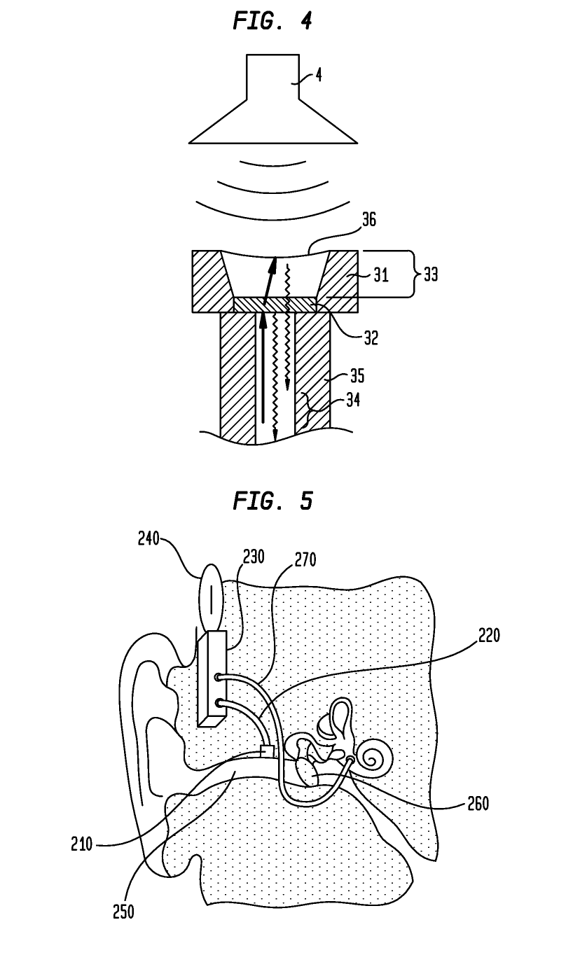

An exemplary embodiment of the present invention includes a sensor corresponding to a Fabry-Perot interferometer, as is depicted by way of example in FIG. 4. In an exemplary embodiment, the interferometer device 3 detailed above in FIG. 2 may be a Fabry-Perot interferometer. The sensor of FIG. 4 comprises two parallel mirrors 32 and 36 separated by an air gap 33. The first mirror 32 is semi-reflective, thus reflecting part of the incident light from the light source (not shown, but may correspond to the light source 1 of FIG. 2) while the other part of the incident light from the light source travels to the second mirror 36. The second mirror 36 is a flexible membrane/diaphragm with a reflective surface at the back (the side of the air gap). When acoustic energy waves, which include sound waves from a source of sound 4 and other energy waves caused by the sound waves, such as energy waves transmitted through the skin/tissue in the case of a subcutaneous sensor, impinge on the mirror 36, the interference pattern of the light traveling back through the fiber optical wave guide 35 as a result of reflection from the second mirror 36 will change. This can be detected by the detector (which may correspond to the detector 6 of FIG. 2, which is not shown in FIG. 4) and sent to the sound processor (which may correspond to sound processor 7 of FIG. 2, which is not shown in FIG. 4).

In FIG. 4, the two mirrors 32 and 36 are held by a base 31 having a cavity forming the air gap 33. The sensor is coupled to the fiber optical waveguide 35 that is attached at the first semi-reflective mirror 32. In an exemplary embodiment, the first mirror 32 may be the polished end face of the fiber optical waveguide 35 (the polished end face forming a semi-reflective mirror). In an exemplary embodiment, the fiber optical waveguide 35 is directly attached at the air gap 33 of the base 31. In an exemplary embodiment, the phase shift between the light reflected at the first mirror 32 and the second mirror 36 changes when the flexible membrane which serves as the second mirror 36 moves due to the acoustic energy waves in the form of sound waves from source of sound 4. As shown in FIG. 4, the air gap 33 may have a conical shape, which, in an exemplary embodiment results in a certain amount of amplification due to the concentration of the sound energy/other energy.

As illustrated in FIG. 5, the sensor 210, which may correspond to the sensor of FIG. 4 and/or other sensors disclosed herein, may be placed in the external part of the ear canal 250 near the tympanic membrane 260. In such an exemplary embodiment, the hearing prosthesis using the sensor 210 may harness a natural amplification of 5-20 dB of the sound/noise, depending on frequency and direction, and the directionality of the outer ear. In the case of a subcutaneously implanted sensor 210, the extremely thin layer of skin/tissue in the ear canal results in a relatively lower amount of attenuation of acoustic waves resulting from source of sound 4 through the skin.

The ear canal 250 is divided into two parts. The cartilaginous part forms the outer third of the canal and contains the cartilage and the continuation of the cartilage framework of the pinna. The bony part forms the inner two thirds. In an exemplary embodiment, the sensor 270 may be fixed in the bony part.

The sensor and/or the fiber optical waveguides detailed herein are made to have diameters smaller than about 4.5 mm (this dimension corresponding to the typical diameters of conventional microphones). In an exemplary embodiment of the present invention, the fiber optical waveguides have an outer diameter of about 120-380 μm, depending on the type of fiber. In an exemplary embodiment, to further decrease the likelihood of necrosis, the surfaces in contact with the skin could be treated such that the skin can more easily attach to those surfaces. Accordingly, embodiments of the present invention result in a relatively reduced likelihood that organs may suffer from adverse affects such as skin necrosis and cholesteatoma, at least in comparison to microphones such as those disclosed in, for example, U.S. Pat. No. 6,697,674, which discloses a microphone that is relatively large, with a diameter of approximately 4.5 mm, and suspended in silicon.

Thus, an embodiment of the present invention includes a hearing prosthesis that utilizes a Fabry-Perot interferometer to in a highly sensitive and small microphone that is adapted to have a typical size in the range of the used fiber optical waveguide, which may have an outer diameter of about 120-380 μm. Thus, a sensor having a diameter in the range of 1 mm or less can be achieved. Due to this small size, irritation of the skin in the ear canal and skin necrosis and cholesteatoma can be at least decreased, relative to larger sensors.

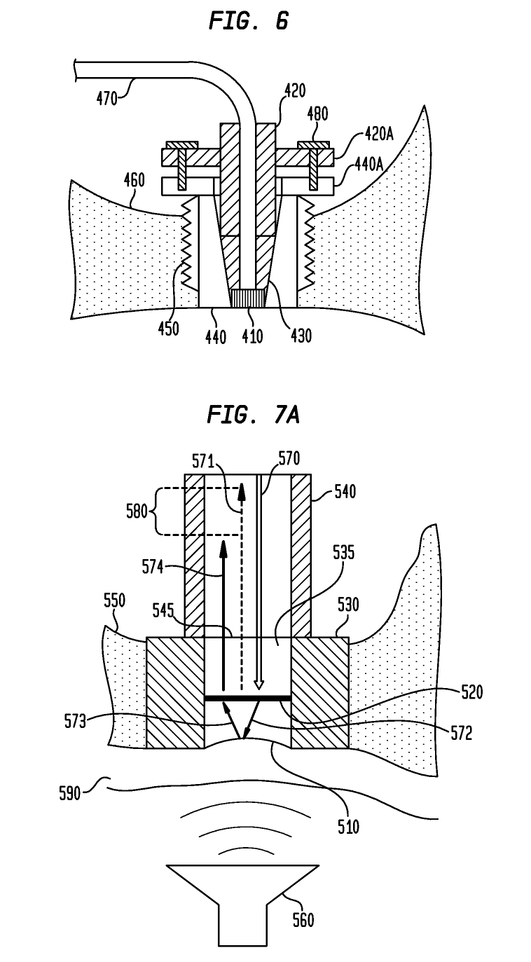

In an embodiment of the present invention, the sensor may be fixed in the bone in a biocompatible fixation structure as illustrated in FIG. 6, which may be a metal biocompatible fixation structure. In an exemplary embodiment, the fixation structure of FIG. 6 may decrease the chances of necrosis, because the sensor is unable to move relative to the skin. In order to at least lessen the likelihood of cholesteatoma, the metal fixation structure may be designed in such a way that the skin cannot grow into the middle ear cavity. In an exemplary embodiment, a hole may be drilled through the mastoid bone 460, in which the fixation structure may be inserted. The fixation structure includes a fixation ring 440 (fixation body) having a screw thread 450 that is screwed into this hole until it is flush with the surface of the mastoid bone 460 facing the ear canal. The inside of the ring may have a conical shape 430 and may match the conically shaped outside of a sensor housing 420. The sensor 410 (interferometer device) and the fiber optical waveguide 470 may be embedded in the sensor housing 420. The sensor housing 420 also may include fixation device 480, corresponding to screws or bolts or rivets, etc., to fix the sensor housing 420 to the fixation ring 440. The fixation device may comprise a first flange 440A provided at a side of the fixation ring 440 opposing the ear canal, which can be connected with a second flange 420A provided at the sensor housing 420 via the screw 480. During insertion into a recipient/build-up of the sensor and fixation structure, the sensor 410 may be lowered into the fixation ring 440 until it is blocked by or otherwise is seated in the conical face 430 of the fixation ring 440. The fixation ring 440 and sensor 410 may match in such a way that the diaphragm (not shown in FIG. 6, reference numeral 36 in FIG. 4) matches in such a way that the diaphragm of the sensor 410 lies flush with the bone surface wherein the skin in the inner ear covers smoothly the bone 460, the fixation ring 440 and the sensor 410. Thus, a tight ceiling between the ear canal and the middle ear and/or other cavities is achieved, which, in some embodiments, at least lessens skin growing into the ear canal.

It is noted that in an exemplary embodiment, embodiments of the fixation structure are not restricted to use with the Fabry-Perot interferometer. Some embodiments of the fixation structure may also be use for any kind of implantable microphone, in order to place the microphone accurately in the bone. In particular, the fixation structure provides a stopping mechanism (e.g. flanges 440A, 420A or conically shaped structures 430 as illustrated in FIG. 6) so that the microphone lies flush with the bone surface so that the skin can cover smoothly the bone, the fixation ring 440 and the microphone.

In an exemplary embodiment, the sensor is placed beneath a layer of skin that is very thin (+/−0.1 mm). In an exemplary embodiment, the sensor may also be placed at a location in the recipient to take advantage of the natural amplification of the sound by the outer ear. In such exemplary embodiments, at least with respect to the optical microphone disclosed herein, the disclosed implantable microphone system may be much less sensitive to unwanted body noise, compared with other subcutaneous microphones.

In an exemplary embodiment, various passive and active body noise cancellation techniques can be applied to the hearing prosthesis as disclosed herein to further reduce the signal to noise ratio of the hearing prosthesis. Various principles of operation of such passive and active body noise cancellation techniques will now be described.

Some of the principles of these noise cancellation techniques are described with respect to FIGS. 7 A and 7 B. The sensor in FIGS. 7A and 7B are similar in principle to the sensor of FIG. 4, and additionally comprises a semi-transparent flexible second mirror 520 in the form of a diaphragm, located between the reflective and flexible first mirror 510, which is also in the form of a diaphragm, and the semi-reflective surface 545. The second mirror 520 is a diaphragm and may have a comparable stiffness as the first diaphragm 510 located against the skin layer 590. Because the fixation device of FIG. 6 is not necessary for explaining the function of this example, the fixation of the sensor in the bone 550 is illustrated in a simplified manner in FIG. 7A and FIG. 7B. In this regard, FIG. 7A and 7B depict the sensor body 530 inserted in a hole of the bone 550 so that no steps between the sensor and the bone 550 occur on the skin 590 side of the bone 550. In the sensor body 530, the flexible reflective first diaphragm 510 and the semi-reflective surface 545 define a cavity 535 in which the second mirror 520 is disposed or otherwise bifurcates, as shown. The second mirror 520 splits an incident light beam 570 into a transmitted light beam 572 and a first reflected light beam 571. At least a portion of the transmitted light beam 572 is reflected by the first diaphragm 510, thus resulting in a second reflected light beam 573. The second reflected light beam 573 is transmitted through the second mirror 520 and propagates back to the main housing (not shown in FIG. 7A and 7B, but may correspond to the main housing 80 or other housings described herein) through fiber optical waveguide 540 as beam 574 and, in an exemplary embodiment, interferes with beam 571 dependent on a phase shift 580.

When sound waves 560 impinge on the skin 590, resulting in the propagation of energy waves across the skin 590 to the first diaphragm 510 (i.e., acoustic energy impinge on the first diaphragm 510), a phase shift 580 between light 573 reflected on the first mirror 510 and the light 571 reflected at the second mirror 520 changes depending on the vibration of the first mirror 510 induced by the sound waves 560. It is noted that the light 574 also produces a phase shift with light reflected at the semi-reflective surface 545, although this is not shown in FIG. 7A. Thus the light intensity of the light traveling through the fiber optical waveguide 540 back to the detector (not shown) in FIGS. 7A and 7B is modulated due to the vibrations of the first diaphragm 510.

In an exemplary embodiment, when the sensor of FIGS. 7A and 7B is accelerated due to bone conducted vibration 555 (i.e., body noise), both the first mirror 510 and the second mirror 520 will be deformed as exemplified by FIG. 7B, whereas external sound 560 only moves the first mirror 510. The differential movement of the first and second mirrors 510 and 520 are due to the vibration of the second mirror 520. This differential movement generates a phase shift 580 which causes intensity modulation of the reflected total light. Thus, the body noise is passively cancelled by the sensor.

In an exemplary embodiment, the passive noise cancellation may be used in combination with active noise cancellation, which may, in some embodiments, further improve signal quality and support speech intelligibility by the recipient and the interpretation of acoustic signals by the recipient. An exemplary principle of the active noise cancellation according to an embodiment of the present invention is the generation of a plurality of electrical signals based on the detected acoustic energy waves (sound waves/energy waves). The different signals are used in an adaptive algorithm to calculate improved signals, wherein the vibration part is removed for driving the hearing prosthesis actuator e.g. the cochlear implant electrodes. Such algorithms may be implemented for example, in the sound processor 70 of FIG. 3, or may be implemented in additional processing units. In some exemplary embodiments, active noise cancellation can be achieved by several measures, as will now be described.

In an exemplary embodiment of active noise cancellation according to an embodiment of the present invention, a polarizing beam splitter may be used to split the light outputted by the light source 1 into at least two beams with different polarization. These two light beams are sent via a fiber optical waveguide to the sensor, which, in an exemplary embodiment, may be similar to the sensor shown in FIGS. 7A and 7B. The sensor according to this exemplary embodiment may include a semi-transparent first mirror, which reflects light of the first polarization direction, while the light with the other polarization is passed through the first mirror and where it is reflected by the second mirror. Both mirrors are flexible diaphragms. The semi-transparent first mirror is a flexible membrane with stiffness comparable to the stiffness of the second diaphragm with skin loading. Thus, the first polarized light beam is used to only measure vibration, while the second polarized light beam measures both sound and vibration. In this exemplary embodiment, the two light beams are sent to two photo detectors, one of which detects the first light beam and the other of which detects the second polarized light beam. The signals of the detectors are used in an adaptive algorithm to cancel out body noise while needing only one light source, one sensor and one fiber optical waveguide.

In an alternative exemplary embodiment of active noise cancellation according to an embodiment of the present invention, instead of utilizing the beam splitter, the semi-transparent diaphragm could be provided with a semi-reflective coating, which reflects only light with a particular polarization. It is also to be noted that for this example, two detectors are not essentially necessary to detect the two polarized light beams independently. A switchable filter may also be used wherein, for example, a liquid crystal filter is switchable between two polarization stages which allow transmission of light of different polarization. In this case, the system might be operated in a pulsed timing mode. Such a configuration may, in an exemplary embodiment, reduce the number of components and reduce electrical power consumption.

In another exemplary embodiment of active noise cancellation according to an embodiment of the present invention, two beams with different wavelengths are used. In such an arrangement, two light sources with different wavelengths may be used or one light source may be used together with a spectral filter such as a prism or a grating, or a color filter integrated in a diaphragm of the sensor.

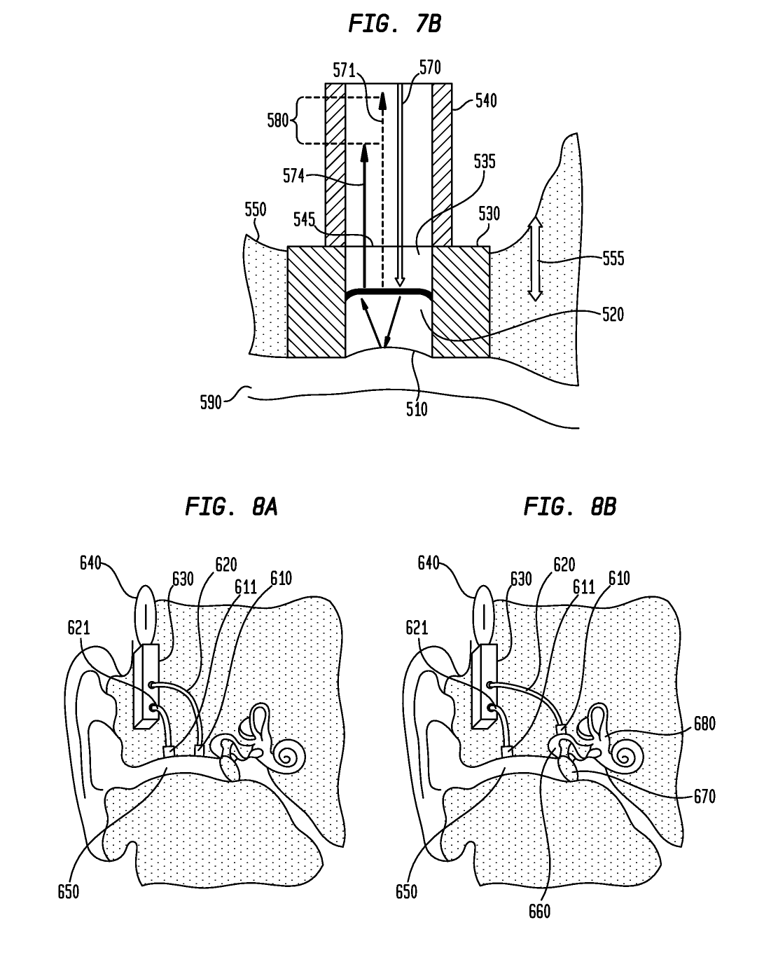

In another exemplary embodiment of active noise cancellation according to an embodiment of the present invention, the light beam can be split into two beams and directed to two different sensors. One of the sensors is sensitive for sound and the other sensor is sensitive for vibration. Examples for vibration sensors are depicted in FIGS. 10A, 10B, 11, 12 and 13, which are described in greater detail below. Implantation configurations with two different sensors are depicted in FIGS. 8A and 8B, which are also described in greater detail below.

In another exemplary embodiment of active noise cancellation according to an embodiment of the present invention, both sensors may be sensitive to both sound and vibration. The two sensors may be adapted to have a phase difference for energy waves resulting from the sound, but not for energy waves resulting from the vibration. In an exemplary embodiment, such may also be used to cancel out the vibration part. For this application, in an exemplary embodiment, two microphones may be positioned in the direction of the sound path and there is enough space between the sensors to practice active noise cancellation. The sensors could be implanted as illustrated, by way of example, in FIGS. 8 A and 8B, as will be described in greater detail below).

In another exemplary embodiment of active noise cancellation according to an embodiment of the present invention, instead of two light beams and/or sensors, more beams and/or sensors may be used. The multiple sensors may all be placed in the ear canal, but also at other locations such as the middle ear, subcutaneous above the pinna and/or combinations thereof. In an exemplary embodiment, the sensor may be placed in the middle ear (as is depicted by way of example in FIGS. 8B, 11, 12 and 13), in the ear canal (as is depicted by way of example in FIGS. 5, 8A and 8B), in the inner ear (as is depicted by way of example in FIGS. 9A), at a subcutaneous location (depicted by way of example in FIG. 9B) or a percutaneous location (depicted by way of example in FIG. 14).

Below, several configurations for the active noise cancellation according to exemplary embodiments are detailed in connection with FIGS. 8A, 8B, 9A, 9B, 10A, 10B, 11, 12, and 13. It is noted that in an exemplary embodiment of the present invention, the configuration according to FIGS. 9A and 9B may also be used in an embodiment utilizing only one sensor.

Referring to FIGS. 8A and 8B, there is shown an exemplary implantation configuration with two sensors. Both FIGs. show an implanted main housing 630 with an antenna 640 for transmitting and/or receiving data and/or energy, and two fiber optical waveguides 620 and 621, which connect a driving and detecting unit within the main housing 630 with two sensors 610 and 611. One of the sensors may be a sensor according to the embodiments detailed herein with respect to FIGS. 4, 7A, and/or 7B. The other sensor may be a vibration sensor according to the embodiments detailed herein with respect to FIG. 10A, 10B, 11, 12 or 13. In FIG. 8A, both sensors are depicted as implanted at the ear canal 650. In FIG. 8B, one sensor 611 is implanted at the ear canal 650, and the other sensor 610 is implanted in the middle ear in general, and by way of example, at the ossicle 660 or the tympanic membrane 670, in particular. In an alternative embodiment, sensor 610 may also be implanted close to the inner ear 680. For example, sensor 610 may be implanted close to the oval window of the cochlea. In an exemplary embodiment, the embodiment associated with FIG. 8B (where a sensor is implanted in the middle ear) takes more advantage of the ear's intrinsic ability of sound amplification.

FIGS. 9A and 9B depict additional exemplary embodiments of a hearing prosthesis with different locations of the sensor 710. Both FIGs. depict an implanted main housing 730 with an antenna 740 for transmitting and/or receiving data and/or energy, and a fiber optical waveguide 720, which connects a driving and detecting unit within the main housing 730 with the sensor 710. FIG. 9A depicts placement of the sensor 710 at the inner ear 780. FIG. 9B depicts placement of the sensor 710 subcutaneously at a location close to the earlobe of the recipient's pinna. The position of the sensors according to the embodiment of FIG. 9A, in some embodiments, is highly effective in terms of sensitivity and resistance against body noise. The position of the sensor according to the embodiment of FIG. 9B is easy accessible.

In an exemplary embodiment, the fixation device of the embodiment of FIG. 6 may be usable in some and/or all of the embodiments of FIGS. 5, 7A, 7B, 8A, 8B, 9A, 9B, 10A and/or 10B

In an exemplary embodiment, the optical sensor depicted in FIGS. 4, 7A and 7B may also be used, in a modification to some of the embodiments described herein, as a vibration sensor as illustrated in FIGS. 10A and 10B. The embodiments of FIGS. 10A and 10B include a base 830 in which a cavity is formed. The base is attached at an end portion of an fiber optical waveguide 840. The cavity includes a semi-transparent first mirror 820 and 870, respectively, and a second mirror 810 and 860, respectively. FIG. 10A depicts an exemplar embodiment where the second mirror 810 may be rigidly placed at a bottom of the cavity 835 in the body 830, and the semi-transparent first mirror 820 is a flexible diaphragm placed within the cavity in the body 830 distant from the first mirror 810. In use of the embodiment of FIG. 10A, body noise 885 is transmitted from the bone 880 to the flexible semi-transparent mirror 820 and deforms the flexible semi-transparent mirror 820. Thus, the sensor is sensitive for vibration.

An exemplary variation of the vibration sensor according to FIG. 10A is depicted in FIG. 10B. In FIG. 10B, the first semi-transparent mirror 870 is located at the interface between the cavity 835 of the body 830 and the end face of the fiber optical waveguide 840. The second mirror 860 is in the form of a flexible beam with a mass 861 attached thereto. The mass of the mass 861 influences the sensitivity of the vibration sensor. Specifically, vibrations cause the second mirror 860 to move, and this movement is influenced in part by the mass of the mass 861 and/or the location of the mass 861 (or, more accurately, the length of the second mirror 860 from the wall of the cavity to the mass 861). In an exemplary embodiment, no mass 861 is present attached to the second mirror 860. Instead, the mass of the mirror 860 is relied on (the mirror 860 does not extend across the cavity, in contrast to the mirror 820 of FIG. 10A). The principle of interference vis-à-vis incident beam 851, transmitted beam 853, first and second reflected beams 852 and 854 is similar to that explained with respect to FIG. 4. These optical vibration sensors may be used in combination with the optical microphones described above with respect to FIGS. 4, 7A, and 7B, in a two-sensor configuration as illustrated in FIGS. 8A and 8B. Such may be done to acquire additional signals for eliminating body noises. These vibration sensors may also be used in a one-sensor configuration as illustrated in FIGS. 9A and 12 in order to measure vibrations of moving parts in the middle or inner ear, such as, by way of example, the ossicles, caused by external sound.

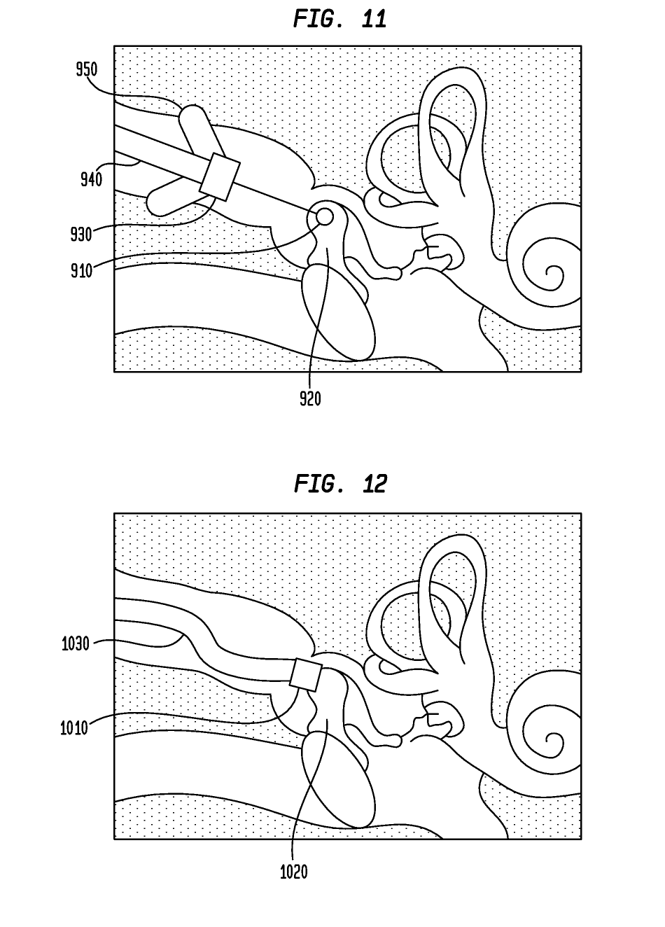

FIG. 11 depicts a modification of the sensor according to the embodiment of FIG. 4, wherein a rod 910 is mounted to or otherwise mechanically coupled to the flexible membrane of the optical sensor 930. The optical sensor 930 is attached to the fiber optical waveguide 940. In an exemplary embodiment, the rod 910 is connected to the ossicles 920 (as exemplified in FIG. 11), the tympanic membrane or the round or oval window. The optical sensor 930 is held in place with respect to the tissue by a fixture 950. In this exemplary embodiment, the advantage of the natural gain of up to ±35 dB of the human ear might be harnessed. In an exemplary embodiment of the invention, the sensor is designed in such a way that it will not significantly restrict the movement of the ossicle chain. In an exemplary embodiment, the rod is well-aligned with the moving part to which it is connected.

FIG. 12 depicts another exemplary embodiment of the present invention, wherein an optical vibration sensor 1010 that is connected to the fiber optical waveguide 1030, such as described in connection with the embodiments of FIGS. 10A and 10B, may be connected to the ossicle 1020, the tympanic membrane and/or the round or oval window. In an exemplary embodiment according to this embodiment, natural gain of up to ±35 dB of the human may be harnessed. In an exemplary embodiment of this embodiment, the sensor may be designed in such a way that it will not restrict the movement of the ossicle chain.

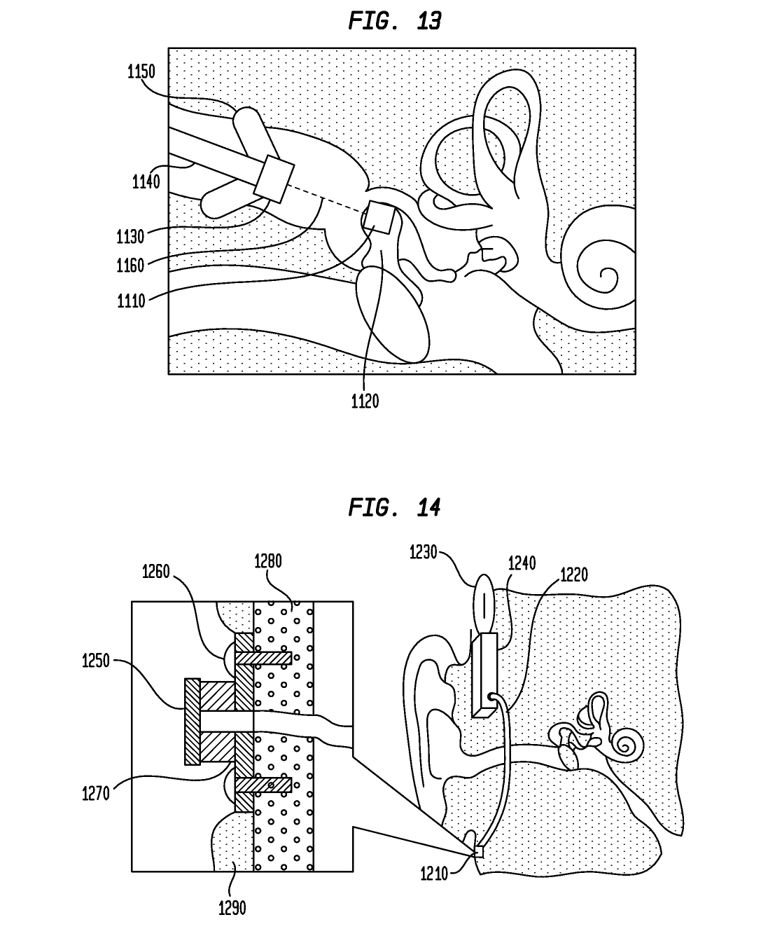

In an exemplary embodiment, in a two-sensor configuration, an optical sensor as described in connection with the embodiments of FIGS. 4, 7A and 7B may be combined with other sensors, such as those described in U.S. Pat. No. 6,491,644. In an exemplary embodiment, an arrangement with a sensor according to U.S. Pat. No. 6,491,644 is exemplified in FIG. 13. In case of combining a sensor configuration as illustrated in FIGS. 4, 7A and 7B with a state of the art sensor according to FIG. 13, in an exemplary embodiment, a system may result which may not be significantly or not at all sensitive to body-induced, bone-conductive vibration. In an exemplary embodiment of such a combination, there may be provided a hearing prosthesis system that may not significantly or at all influence the movement of the ossicle chain. In an exemplary embodiment, the design and placement of the fixation of both the sensor and the refractive surface on the moving parts is such that they are well-aligned.

In an exemplary embodiment, as an alternative to one light source, two or multiple light sources may also be used. Instead of a VCSEL, other types of light sources having sufficient coherence lake laser diodes can be used. LEDs can also be used, or other types of light sources which can be coupled into a fiber optical waveguide, and which use limited power. The light sources may be of two different polarizations or of two or multiple wavelengths. Instead of a Fabry-Perot sensor, other type of sensors can also be used. In its simplest form, only one reflecting diaphragm can be used to reflect the light. The difference in path length is then used to detect the sound. Other types of interferometers can also be used, such as a Michelson interferometer or a Mach-Zehnder interferometer, Bragg grating, etc. Also, different kinds of lenses and mirrors can be used, such as, for example, a Fresnel lens. All these solutions can be used to realize the concepts disclosed in this patent application.

In an exemplary embodiment, a sensor as disclosed herein may be used as a percutaneous microphone placed, for example, behind the earlobe of the recipient's pinna, as is illustrated in FIG. 14. FIG. 14 depicts an implanted main housing 1230 with an antenna 1240 for transmitting and/or receiving data and/or energy, and a fiber optical waveguide 1220, which connects a driving and detecting unit within the main housing 1230 with the sensor 1210. In FIG. 14, the sensor 1210 contains the interferometer device 1250 and protrudes from the skin 1290. The interferometer device 1250 may be fixed in the bone 1280 through a fixture 1270 that is held by screws 1260. In an exemplary embodiment of this embodiment, there may be limited attenuation and limited body noise sensitivity, because there is no skin covering the interferometer device 1250.

As noted above, additional features of the cochlear implant 100 will now be described with reference to functional diagrams of the cochlear implant 100 according to an embodiment of the present invention. Cochlear implants having these features may, in exemplary embodiments, be utilized with cochlear implant 100 will now be described. It is noted that while cochlear implant 100 has been disclosed as a totally implantable cochlear prosthesis, embodiments of the implantable microphone may be practiced with a cochlear implant that is not totally implantable.

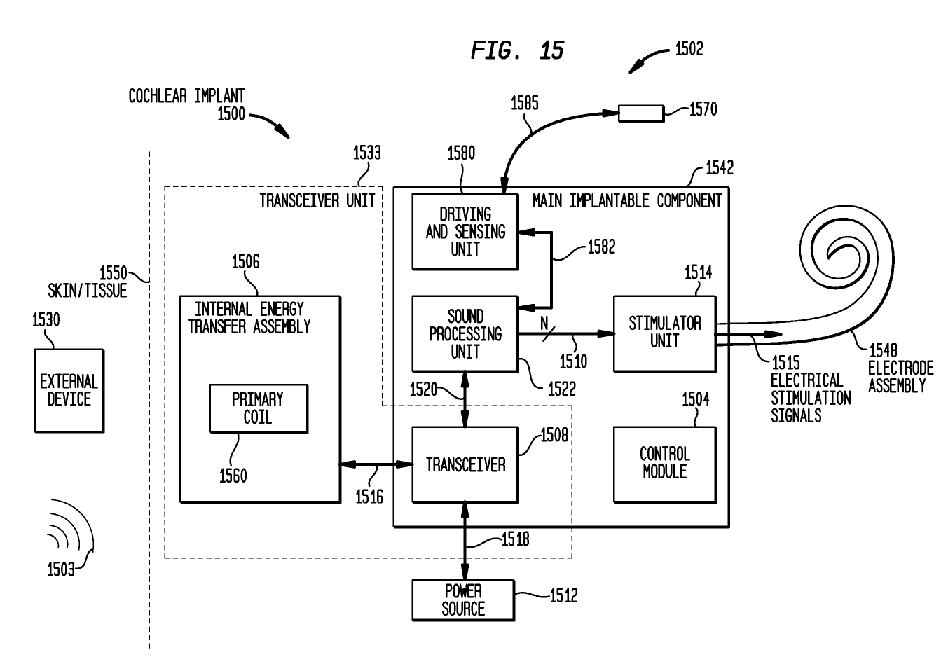

FIG. 15 is a functional block diagram of embodiments of cochlear implant 100 in which embodiments of the present invention may be implemented, referred to as cochlear implant 1500 herein. Similar to the above embodiments, cochlear implant 1500 is totally implantable; that is, all components of cochlear implant 1500 are configured to be implanted under skin/tissue 1550 of a recipient. Because all components of cochlear implant 1500 are implantable, cochlear implant 1500 operates, for at least a period of time, without the need of an external device, such as external device 1530.

Cochlear implant 1500 comprises a transceiver unit 1533, a main implantable component 1542, a rechargeable power source 1512, and an electrode assembly 1548. The embodiments of FIG. 15 are illustrative, and it would be appreciated that one or more components may be disposed in the same or different housings.

As shown in FIG. 15, transceiver unit 1533 comprises an internal energy transfer assembly 1506 and a transceiver 1508. As discussed below, internal energy transfer assembly 1506 and transceiver 1508 cooperate to receive power and/or data from external device 1530. As used herein, transceiver unit 1533 refers to any collection of one or more implanted components which form part of a transcutaneous energy transfer system. Furthermore, internal energy transfer assembly 1506 refers to an assembly which includes the component(s) of a transceiver unit 1533 which transmit and receive data, such as, for example a coil for a magnetic inductive arrangement, an antenna for an alternative RF system, capacitive plates, or any other suitable arrangement. As such, in embodiments of the present invention, various types of energy transfer, such as infrared (IR), electromagnetic, capacitive and inductive transfer, may be used to transfer the power and/or data from external device 1530 to cochlear implant 1500.

In the illustrative embodiments of FIG. 15, inductive transfer is used to transfer power and/or data, and internal energy transfer assembly 1506 comprises a primary coil 1560. Transceiver 1508 is connected to primary coil 1560 and comprises the circuit elements used to decode and distribute the received power and/or data. The electrical connection between primary coil 1560 and transceiver 1508 is illustrated by bidirectional arrow 1516. In the embodiments of FIG. 15, transceiver 1508 is physically disposed in main implantable component 1542.

As shown, cochlear implant 1500 further comprises main implantable component 1542. Main implantable component 1542 includes transceiver 1508 and sound processing unit 1522. Main implantable component 1542 further includes stimulator unit 1514 and control module 1504.

As shown, internal energy transfer assembly 1506 comprises an implantable microphone system 1502. Implantable microphone system 1502 comprises a sensor 1570, which may correspond to the sensors disclosed herein, including the interferometers, positionable at a measuring point and a driving and sensing unit 1580 located in the main implantable component 1542. It will be understood that in various exemplary embodiments of the present invention, the implantable microphone system 1502 may correspond to any of the microphone systems and/or vibration sensing systems disclosed herein. The driving and sensing unit 1580 is in light communication with the sensor 1570 by fiber optic waveguide 1585. The implantable microphone system 1502 is configured to sense a sound signal 1503. The driving and sensing unit 1580 may include a light source corresponding to light source 1 detailed above with respect to FIG. 2 and may include a detector corresponding to detector 6 detailed above. The driving and sensing unit 1580 converts the light reflected from sensor 1570 into an electrical signal which is delivered to sound processing unit 1522 as disclosed above, in order to enhance hearing. Specifically, pre-processed microphone output in the form of an electrical signal (based on the received reflected light by the detector 6 in the driving and sensing unit 1580) is provided to sound processing unit 1522 via electrical connection 1582. Sound processing unit 1522 implements one or more speech processing and/or coding strategies to convert the pre-processed microphone output into data signals 1510 which are provided to a stimulator unit 1514. Based on data signals 1510, stimulator unit 1514 generates electrical stimulation signals 1515 for delivery to the cochlea of the recipient. In the embodiment illustrated in FIG. 15, cochlear implant 1500 comprises an embodiment of electrode assembly 118 of FIG. 1, referred to as electrode assembly 1548, for delivering stimulation signal 1515 to the cochlea.

Cochlear implant 1500 also includes rechargeable power source 1512. Power source 1512 may comprise, for example, one or more rechargeable batteries. As noted above, power is received from external device 1530, and is distributed immediately to desired components, or is stored in power source 1512. The power may then be distributed to the other components of cochlear implant 1500 as needed for operation.

As noted, main implantable component 1542 further comprises control module 1504. Control 1504 includes various components for controlling the operation of cochlear implant 1500, or for controlling specific components of cochlear implant 1500. For example, controller 1504 may control the delivery of power from power source 1512 to other components of cochlear implant 1500.

For ease of illustration, internal energy transfer assembly 1506, main implantable component 1542 and power source 1512 are shown separate. It would be appreciated that one or more of the illustrated elements may be integrated into a single housing or share operational components. For example, in certain embodiments of the present invention, internal energy transfer assembly 1506, main implantable component 1542 and power source 1512 and driving and sensing unit 1580 may be integrated into a hermetically sealed housing, and

It is noted that the sensors disclosed herein may be optical sensors/optical microphones as disclosed herein.

Exemplary embodiments of the present invention include optical sensors that are relatively small, having a diameter of about 0.5 mm or less, and are, thus, aesthetically satisfying. In an exemplary embodiment, potentially bio-toxic materials may be hermetically sealed in the main implant 1230, and that the sensor 1210 may be bio-safe. Should the sensor be damaged as the result of an impact, there will be no safety risk to the recipient as a result of the intermingling of bio-toxic materials with body tissue and/or body fluids. In an exemplary embodiment, the sensitive and vulnerable sensor part may be easily removed and/or covered during activities which might harm the sensor. The sensor part may be replaced when necessary.

In an exemplary embodiment, there is an optical microphone that includes a passive bio-compatible sensor part located at a measurement location which is connected by a bio-compatible fiber optical waveguide to a housing of a main implant, which may be located at various locations within a recipient. The sensor and the fiber optical waveguide may be relatively small and may provide for flexibility with respect to placement in the recipient, with good signal-to-noise ratio and other clinical advantages. In an exemplary embodiment, the sensor may be placed in the ear canal where it can take benefit of the natural amplification of the outer ear. When placed in the middle ear or inner ear, however, the natural gain might be, in some embodiments, even higher. In an exemplary embodiment, no electrical leads and/or no analog/digital converters between sensor and housing are present. In an exemplary embodiment, this makes the implantable hearing prosthesis utilizing the system safer and more energy efficient. In an exemplary embodiment, the sensor is bio-compatible, even when the diaphragm of the sensor is disrupted due to impact. In an exemplary embodiment, the sensor has a high resolution and may be used in variations as an input for an adaptive algorithm to cancel out body noise. In an exemplary embodiment, due to the small size of the sensor, the system is less sensitive to skin necrosis when used subcutaneously. The fixation method disclosed herein, in an exemplary embodiment, may reduce the risk of cholesteatoma. In an exemplary embodiment, the light source can be used in pulsed operation, thus reducing the power consumed by the hearing prosthesis. In an exemplary embodiment, the light source detector and all associated electronics are relatively small and may be placed on the same printed circuit board.

In an exemplary embodiment of the present invention, the implantable microphone system including the optical microphone as detailed herein may be less prone to bone conducted noises than the device of U.S. Pat. No. 6,697,674 that uses an acoustic transducer. In an exemplary embodiment of the present invention, the implantable microphone system including the optical microphone as detailed herein may be less prone to scattering and absorption of the reflected light in the inner ear than the implanted microphone of U.S. Pat. No. 6,491,644 teaches another example of an implanted microphone.

The invention described and claimed herein is not to be limited in scope by the specific preferred embodiments herein disclosed, since these embodiments are intended as illustrations, and not limitations, of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

What is claimed is:

1. A hearing prosthesis, comprising:
an implantable housing containing a detector;
a light source;
a fiber optical waveguide extending from the implantable housing in light communication with the light source and the detector; and
an interferometer connected to the fiber optical waveguide and located outside of the implantable housing, the interferometer being in light communication with the fiber optical waveguide, the light source and the detector,
wherein the detector is configured to convert a light signal indicative of acoustic energy waves that impinge upon the interferometer into a signal indicative of the acoustic energy waves.

2. The hearing prosthesis of claim 1, wherein the interferometer is a Fabry-Perot interferometer.

3. The hearing prosthesis of claim 1, wherein the interferometer comprises:
a base with a cavity that is in light communication with the fiber optical waveguide;
a semi-reflective surface that forms a first boundary of the cavity; and
a reflective surface that forms a second boundary of the cavity, the reflective surface being spaced apart from the semi-reflective surface,
wherein the reflective surface is a flexible diaphragm configured to flex in response to the acoustic energy waves.

4. The hearing prosthesis of claim 3, wherein the semi-reflective surface is in contact with the fiber optical waveguide and is located at a first end of the cavity, and wherein the reflective surface is located at a second end of the cavity opposite the first end of the cavity.

5. The hearing prosthesis of claim 3, wherein:
the interferometer further comprises a flexible and semi-reflective diaphragm spaced apart from the semi-reflective surface and the reflective surface;
the hearing prosthesis is configured to direct at least a first and second light beam having different properties through the fiber optical waveguide to the interferometer; and
the semi-reflective diaphragm is adapted reflect more of the light of the first beam than the light of the second beam.

6. The hearing prosthesis of claim 3, wherein:
the reflective surface is a rigid surface located at a bottom of the cavity in the body;
the semi-reflective surface is a flexible diaphragm located within the cavity in the body away from the reflective surface; and
the interferometer is configured to sense vibration.

7. The hearing prosthesis of claim 1, wherein:
the interferometer includes a mass;
the semi-reflective surface is located at the interface between the cavity of the body and the end face of the optical fiber,
the reflective surface is a flexible beam with the mass attached thereto, and
the interferometer is a vibration sensor.

8. The hearing prosthesis of claim 1, comprising:
at least two fiber optical waveguides;
at least two interferometers in light communication with respective fiber optical waveguides; and
a processing unit configured to actively eliminate body sound detected by the least two interferometers.

9. The hearing prosthesis of claim 5, wherein the properties of the at least first and second light beams are selected from the group consisting of wavelength and polarization.

10. The hearing prosthesis of claim 1, wherein the hearing prosthesis is a totally implantable hearing prosthesis.

11. The hearing prosthesis of claim 1, wherein the hearing prosthesis includes a fixation device adapted to mount at least one of the interferometer and end of the fiber optical waveguide in a skull bone, wherein the fixation device comprises:
a first body with an external thread screwable into a skull bone and having a conically shaped through hole; and
a second body adapted to be secured to the first body such that the second body fits into the conically shaped through hole of the first body,
wherein the interferometer and the end of the fiber optical waveguide are embedded in the second body.

12. The hearing prosthesis of claim 1, wherein the implantable housing is configured to hermetically seal the detector in the implantable housing.

13. An implantable Fabry-Perot interferometer configured for use in at least one of a microphone and a vibration sensor, comprising:
a base with a cavity;
a semi-reflective surface that is located in the cavity; and
a reflective surface that forms a boundary of the cavity, the reflective surface being spaced apart from the semi-reflective surface;
wherein at least one of the semi-reflective surface and the reflective surface is a flexible diaphragm.

14. The implantable Fabry-Perot interferometer of claim 13, wherein:
the semi-reflective mirror is connected to an end of a fiber optical waveguide and is located at a first end of the cavity; and
wherein reflective surface is flexible and is receptive for sound energy and is located at a second end of the cavity.

15. The implantable Fabry-Perot interferometer of claim 14, further comprising:
a flexible and semi reflective second diaphragm, wherein the second diaphragm is adapted to reflect more or less of a first light beam than a second light beam having different properties than the first light beam.

16. The implantable Fabry-Perot interferometer of claim 13, wherein the cavity has a conical shape with the first end of the cavity having a smaller diameter than the second end of the cavity.

17. The implantable Fabry-Perot interferometer of claim 13, wherein:
the base with a cavity is adapted to communicate with an end of a fiber optical waveguide;
the reflective surface is a rigid surface located at a bottom of the cavity in the body; and
the semi-reflective surface is a flexible diaphragm placed within the cavity in the body distant from the reflective surface.

18. The implantable Fabry-Perot interferometer of claim 13, wherein:
the base with a cavity is in light communication with an end face of a fiber optical waveguide;
the semi-reflective surface is located at the interface between the cavity of the body and the end face of the optical fiber; and
the interferometer includes a mass, wherein the reflective surface is a flexible beam with the mass attached to an end thereof.

19. An implantable fixation device adapted for mounting an implantable microphone in a skull bone, wherein the fixation device comprises:
a first body with an external thread adapted to be screwable into the skull bone and having a conically shaped through hole; and
a second body adapted to be secured to the first body, which has a conically shaped end portion, dimensioned to fit into the conically shaped through hole of the first body,
wherein an implantable microphone is connected to the second body.

20. The implantable fixation device of claim 18, wherein the implantable microphone is at least one of an interferometer and a fiber optical waveguide, wherein one of the interferometer and an end of the fiber optical waveguide are embedded in the second body.
